(19) United States
(12) Patent Application Publication
(10) Pub. No.: US 2026/0061411 A1
(43) Pub. Date: Mar. 5, 2026

(54) SYSTEMS AND METHODS FOR SUSTAINING OPTIMAL PHOTOCATALYSIS PERFORMANCE

(71) Applicant: Bioleum Corporation, Oklahoma City, OK (US)

(72) Inventor: Craig A. Grimes, Raleigh, NC (US)

(21) Appl. No.: 19/382,477

(22) Filed: Nov. 7, 2025

Related U.S. Application Data
(63) Continuation of application No. 17/752,516, filed on May 24, 2022, now abandoned.
(60) Provisional application No. 63/193,803, filed on May 27, 2021.

(51) Int. Cl. B01J 38/02; B01J 35/39; C07C 1/02

(52) U.S. Cl. CPC B01J 38/02; B01J 35/39; C07C 1/02; B01J 2204/002; B01J 2204/005; B01J 2208/00548

(57) ABSTRACT

A photoreactor having computer actuated input/output ports is operated by introducing reactant through an input port and collecting product through an output port, and upon closure of the input and output ports, treating photocatalyst within the photoreactor to remove intermediates limiting performance of the photocatalyst. Once the photocatalyst is regenerated, introduction of reactant to the photoreactor through the input port and collection of product from the output port can be resumed. The automated process does not require removal of catalyst from the photoreactor and significantly improves process economics.

### CROSS REFERENCE TO RELATED APPLICATIONS/INCORPORATION BY REFERENCE STATEMENT

This application is a continuation of U.S. application Ser. No. 17/752,516, filed May 24, 2022; which claims priority under 35 USC § 119 (e) to U.S. Provisional Application No. 63/193,803, filed May 27, 2021. The entire contents of each of the above-referenced patent applications are hereby expressly incorporated herein by reference.

### BACKGROUND

### 1. Technical Field

The present disclosure relates to systems and methods for photocatalysis and, more particularly, to systems and methods of operating a photoreactor to improve or optimize system performance.

### 2. Brief Description of Related Art

Photocatalytic reduction of CO₂ is a complex, multistep process that combines different aspects of light harvesting, charge separation and transfer, and surface science, with overall conversion efficiency determined, in part, by light absorption properties of the semiconductor, electron and hole transport to surface reaction sites, reactant absorption, catalytic reactions, and product desorption.

Given the ability of a semiconductor photocatalyst to absorb electromagnetic radiation and generate, in turn, an electron-hole pair, photocatalyst efficiency is significantly impacted by the ability of the radiation-generated electrons and holes (1) to avoid unwanted recombination; and, (2) to promote specific reaction steps. For example, the photocatalytic conversion of CO₂ to fuel requires multiple electron transfers that can lead to the formation of many different products. Formation of the different products may depend upon the nature of incident radiation (e.g., intensity and wavelength), as well as the number and direction of transferred electrons, by which the final oxidation state of the carbon atom is determined. For example, photocatalytic conversion of CO₂ in the presence of water vapor may lead to different products arising at the same time including carbon monoxide, methane, and ethane, as well as photocatalyst deactivating carboxylic acid salts or formic acid.

Photocatalyst deactivation is a known problem with semiconductor CO₂ reduction photocatalysts. Under illumination, high performance CO₂ reduction photocatalysts generate copious amounts of electrons and holes, leading to formation of both desired and undesired products. Active photocatalytic materials are known to partially or even completely deactivate with time during operation due to the formation of intermediate compounds that remain on the surface of the photocatalyst preventing adsorption of reactant molecules. Carboxylates, sometimes referred to as carboxylic acid salts, are a common and undesired deleterious reaction product. Carboxylates over time may block active sites of the semiconductor photocatalyst surface deactivating the photocatalyst.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIGS. 1A and 1B illustrate, respectively, a graphical representation of photoreduction of CO₂ and water vapor to methane and ethane yield, and sunlight-to-fuel conversion efficiency, using a flow-through photoreactor under AM 1.5 illumination. In particular, FIG. 1A is an illustrative graphical representation of a stability test of a Cu_{1.00%}-Pt_{0.35%}—reduced blued-colored titania photocatalyst sample wherein after each test cycle the photocatalyst was physically removed from the photoreactor and exposed to a mild vacuum at 100° C. for 2 h.

FIG. 1B illustrates differential time-dependent (0.5 h increments) Joule (sunlight input) to Joule (hydrocarbon fuel output) photoconversion efficiency of the reduced blue-colored titania photocatalyst sample shown in FIG. 1A sensitized with nanoparticles Cu_{1.00%}-Pt_{0.35%}, where % values indicate weight percent of co-catalyst metal nanoparticles.

FIG. 2 is a schematic drawing of an exemplary photoreactor in accordance with the present disclosure.

FIG. 3 is a block flow diagram of an exemplary process for photocatalytic treatment of CO₂ and water to generate hydrocarbon fuels utilizing a gas chromatograph and a computer to control regeneration of the photocatalyst within the photoreactor.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary and not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, and/or methods disclosed herein can be made and executed without undue experimentation in light of the present disclosure. While the compositions, assemblies, systems, and methods of the present disclosure have been described in terms of particular embodiments, it will be apparent to those of skill in the art that variations may be applied to the compositions and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The present disclosure relates generally to methods and systems for improving performance of a photoreactor. In one non-limiting embodiment, a photoreactor includes computer actuated input/output ports, and is operated by introducing reactant to the photoreactor through an input port and collecting product from the photoreactor through an output port. When it is determined that photocatalyst within the photoreactor requires treatment, the input and output ports are closed and the photocatalyst is treated to remove intermediates limiting performance of the photocatalyst. After treatment of the photocatalyst to remove the intermediates, introduction of reactant to the photoreactor through the input port and collection of product from the photoreactor through the output port are resumed.

In one non-limiting embodiment, a method for photoconversion of CO₂ and H₂O vapor reactants to hydrocarbon fuel product includes the steps of introducing the CO₂ and H₂O vapor reactants into the photoreactor and collecting hydrocarbon fuel product from the photoreactor using at least two computer or microprocessor controlled input/output ports; and then varying the relative CO₂/H₂O concentration into the photoreactor as a function of time, or modulating the temperature or gas pressure as a function of time, to maintain optimal or near optimal photocatalyst performance.

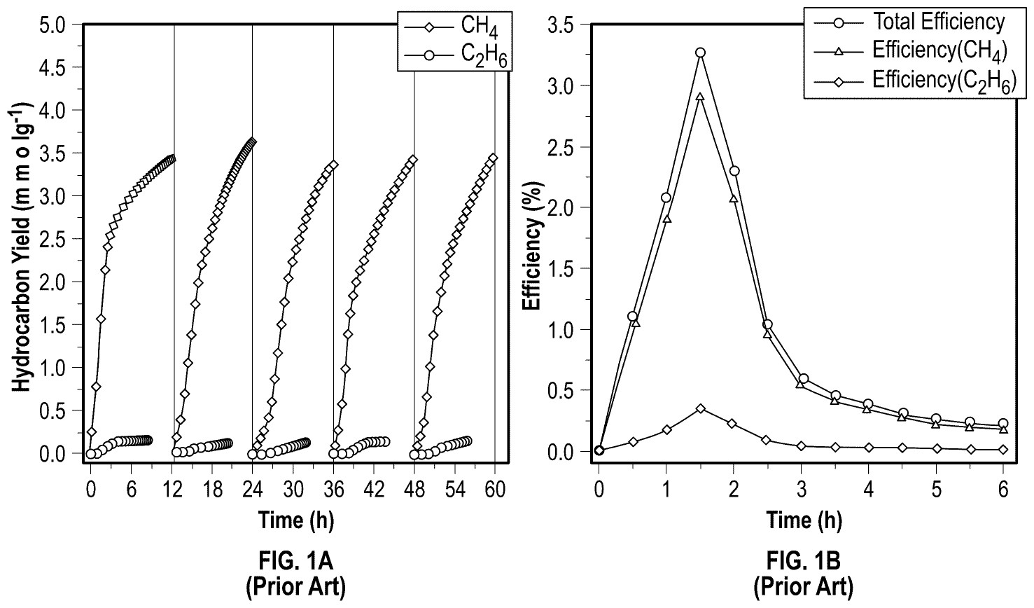

Turning now to the drawings, FIGS. 1A and 1B are graphical illustrations of the performance of an active photocatalyst during photoreduction of CO₂ and water vapor to methane and ethane using a flow-through photoreactor under AM 1.5 illumination. In particular, FIG. 1A is a graphical representation of a stability test of a Cu1.00%-Pt0.35%—containing reduced blued-colored titania photocatalyst sample wherein after each test cycle the photocatalyst was physically removed from the photoreactor and exposed to a mild vacuum at 100° C. for 2 h; % values indicate atomic weight percent. FIG. 1B illustrates differential time-dependent (0.5 h increments) Joule (sunlight input) to Joule (hydrocarbon fuel output) photoconversion efficiency of the Cu1.00%-Pt0.35%—reduced blued-colored titania photocatalyst sample shown in FIG. 1A. The photocatalyst demonstrates an initial stage where performance first increases with time generally due to the increased participation and contribution of reaction sites, and heating of the photocatalyst by the absorption of incident electromagnetic energy of infrared wavelength. This initial stage is followed by a deactivation stage wherein the operational performance continues to decline with time due to formation of unwanted intermediates that block photocatalytically active reaction sites. While it is possible to physically remove the photocatalyst from the photoreactor and manually process the photocatalyst (e.g., heating in a vacuum, washing with deionized water), to regain high or optimal sample performance, manual removal and cleaning of the photocatalyst is time consuming and a costly operation. Described herein are systems and methods to provide modified photoreactor configuration and associated processes that enable sustained operation without an operator having to physically remove the photocatalyst from the photoreactor, clean or refresh the photocatalyst, and then re-install the photocatalyst into the photoreactor.

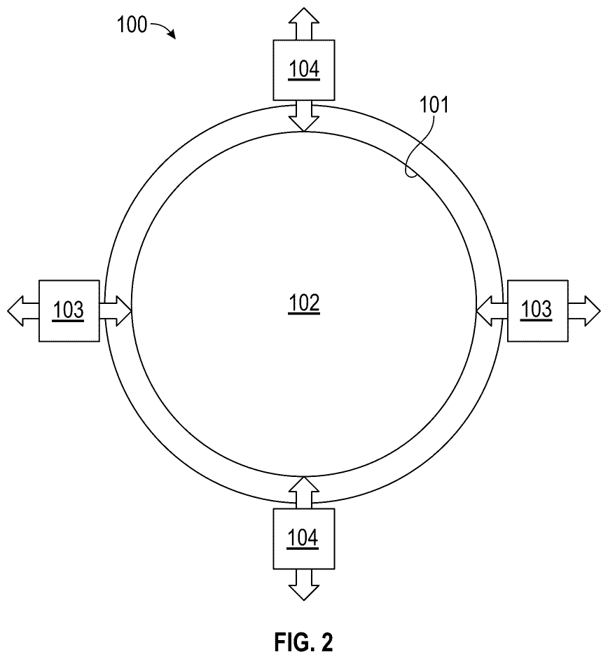

FIG. 2 illustrates an exemplary photoreactor 100 having reactant/product ports 103 (e.g., gas inlet (reactant) and gas outlet (product) ports). The photoreactor includes additional inlet and outlet venting ports 104. Housing 101 of the photoreactor 100 may be formed of stainless steel or other suitable metals (e.g., glass or quartz). The photoreactor 100 may also include a window 102. The window 102 may be optically transparent to allow passage of electromagnetic radiation by which the photocatalyst is illuminated. It is understood that directional gas flow through the photoreactor can be controlled by relative pressure differences between ports.

In some embodiments, to sustain optimal or near-optimal photocatalyst operation, a heating stage may be integrated into the photoreactor 100. In some embodiments, the photoreactor 100 may be positioned on the heating stage. Periodic heating of the photocatalyst via the heating stage, with or without the input reactant flow stopped, may be used to thermally reduce and/or desorb unwanted intermediate products (e.g., carboxylates), from the surface of the photocatalyst. Depending upon the product desorbed from the photocatalyst surface, a function of the input reactant(s), illumination wavelength and intensity, and photocatalyst composition, the photocatalyst can be heated with input/output ports 103 either open or closed and, respectively, venting ports 104 closed or open.

In some embodiments, air or nitrogen may be flowed over the photocatalyst while it is being heated for photocatalyst performance recovery. Reactant/product ports 103 may be closed and venting ports 104 used to introduce reactant air or nitrogen and carry away the output product(s) formed by photocatalyst regeneration.

In some embodiments, the photocatalyst temperature may be raised, independent of the reactant/product (input/output) gas flow, to recover photocatalyst performance. The product outputs from the cleaning step, such as a type of fuel (e.g., methane) may be provided. Ports 104 can remain closed with input (reactant) output (product) ports 103 open.

In some embodiments, ports 103 can be closed and air introduced into the photoreactor 100 by use of ports 104 during which the photocatalyst sample is exposed to ultraviolet illumination. High-energy UV photons may promote decomposition of unwanted intermediates blocking the photocatalyst adsorption sites.

In some embodiments, one or more steps of the method of photocatalyst sample recovery may be automatic. Automation of the photocatalyst sample recovery may provide for automatic operation by which optimal photocatalytic performance is achieved. For example, in some embodiments, the system may cycle through the photocatalyst recovery procedure every 30 minutes with each photocatalyst recovery procedure being, for example 60 seconds, by which optimal photocatalyst performance is maintained.

In some embodiments, the photoreactor 100 may include two or more computer actuated ports 103 and/or 104. In some embodiments, the at least two computer actuated ports may provide for introduction of reactant and collection of product. In some embodiments, upon closure of the at least two computer actuated ports, the photocatalyst at ambient temperature may desorb unwanted intermediates limiting photocatalytic performance. In some embodiments, upon closure of the at least two computer actuated ports, the photocatalyst at ambient temperature may desorb unwanted intermediates limiting photocatalytic performance upon exposure of the photocatalyst to ultraviolet wavelength electromagnetic energy. In some embodiments, upon closure of the at least two computer actuated ports, the photocatalyst at ambient temperature may desorb unwanted intermediates limiting photocatalytic performance upon heating of the photocatalyst.

In some embodiments, the photoreactor 100 may include three or more computer actuated ports 103 and/or 104 configured to provide introduction of reactant and collection of product. In some embodiments, the at least three computer actuated ports may provide for programmed iteration of system operation alternating between photocatalytic conversion of input reactants to desired output products and one or more processes for photocatalyst cleaning and/or recovery. In some embodiments, the at least three computer actuated ports may provide for programmed iteration of system operation alternating between photocatalytic conversion of input reactants to desired output products and photocatalyst cleaning by introduction of one or more liquids to remove unwanted intermediates.

In some embodiments, upon closure of at least two computer actuated ports 103 and/or 104, the photocatalyst at ambient temperature may be exposed to a vacuum for removal of unwanted intermediates from the surface of the photocatalyst. In some embodiments, upon closure of at least two computer actuated ports 103 and/or 104, the photocatalyst may be exposed to a vacuum while illuminated with electromagnetic energy for the breakdown and removal of unwanted intermediates limiting photocatalyst performance. In some embodiments, upon closure of at least two computer actuated ports 103 and/or 104, the photocatalyst may be exposed to a vacuum while illuminated with ultraviolet light for the breakdown and removal of unwanted intermediates limiting photocatalyst performance.

In some embodiments, the photoreactor 100 may include four or more computer actuated ports 103 and/or 104 allowing for introduction of reactant and collection of product. In some embodiments, upon closure of at least two computer actuated ports 103 and/or 104, a gas-phase atmosphere may be passed across the ambient-temperature photocatalyst sample for removal of unwanted intermediates limiting photocatalyst performance. In some embodiments, upon closure of at least two computer actuated ports 103 and/or 104, a gas-phase atmosphere may be passed across the ambient-temperature photocatalyst sample in addition to heating of the photocatalyst sample for removal of unwanted intermediates limiting photocatalyst performance. In some embodiments, upon closure of at least two computer actuated ports, a gas-phase atmosphere may be passed across the ambient-temperature photocatalyst sample in addition to exposure of the photocatalyst sample to electromagnetic energy for removal of unwanted intermediates limiting photocatalyst performance.

Depending upon the input reactant(s), illumination wavelength and intensity, and photocatalyst composition, it may not be necessary to heat the photocatalyst above ambient temperature. The adsorption-site blocking intermediate compounds may simply degas as a function of time. The products obtained from photocatalyst desorption may be useful, in which case they can be collected using product output port 103, or not useful in which case reactant/product ports 103 may be closed and venting ports 104 opened to allow venting of the desorbed degassed compounds from the photocatalyst.

In some embodiments, periodically during operation, reactant-input/product-output ports 103 can be closed, for example through the operation of a computer controlled actuator, and a single port 104 or multiple ports 104 opened exposing the photocatalyst to a vacuum to promote desorption of unwanted intermediates from the photocatalyst that limit photocatalyst operation. This procedure can be done with the photocatalyst at ambient temperature, or this procedure can be done in combination with heating of the photocatalyst by which desorption is promoted. This procedure can also be done in combination with exposure of the photocatalyst to ultraviolet light with the high-energy photons being more readily able to break down unwanted intermediates blocking the photocatalyst adsorption sites, with or without external heating of the photocatalyst. In some embodiments, all ports to the photoreactor may be computer actuated allowing for autonomous operation.

In some embodiments, during photocatalytic conversion of CO₂ and water vapor to hydrocarbon fuels, it may be possible depending upon photocatalyst illumination wavelength and intensity, and photocatalyst composition, to regain optimal or near optimal photocatalyst performance by periodic variation of the relative H₂O/CO₂ amounts in the input reactant stream. Removing either hydrogen or carbon ions from the input stream may directly impact intermediate formation, potentially giving way to removal. Such variation of H₂O/CO₂ input reactant can be used in combination with photocatalyst heating, or exposure of the photocatalyst to high energy photons, (e.g., ultraviolet light), or exposure of the photocatalyst to vacuum. It may also be found that shifting the input reactant flow to either a high or low humidity regime, in combination with a gaseous cross-flow from the venting ports 104 may facilitate the lifting and removal of the unwanted intermediates.

In some embodiments, wherein the photoreactor 100 is used for the photoconversion of CO₂ and H₂O (vapor) to hydrocarbon fuels for example, the photoreactor 100 may include at least two computer or microprocessor controlled input/output ports 103 allowing for introduction of reactant and collection of product. In some embodiments, the relative CO₂/H₂O concentration into the photoreactor 100 may be varied as a function of time to maintain optimal or near optimal photocatalyst performance. In some embodiments, the temperature of the photocatalyst within the photoreactor is modulated as a function of time to maintain optimal or near optimal photocatalyst performance.

In some embodiments, the photoreactor 100 may be used for the photoconversion of CO₂ and H₂O (vapor) to hydrocarbon fuels. The photoreactor 100 may include at least two computer or microprocessor controlled input/output ports 103 allowing for introduction of reactant and collection of product and a computer controlled mass flow controller enabling introduction of reactant and collection of product. The gas pressure within the photoreactor may be modulated as a function of time to promote desorption of unwanted intermediate products from the photocatalyst to enable optimal or near optimal rates of product formation. In some embodiments, the gas pressure within the photoreactor can be modulated as a function of time while the gaseous flow rate through the photoreactor is kept constant. In some embodiments, the gaseous flow rate through the photoreactor is kept constant while the gas pressure within the photoreactor is modulated as a function of time.

In some embodiments, the photoreactor 100 may include at least three computer or microprocessor controlled input/output ports 103 providing for introduction of reactant and collection of product. Upon closure of at least two ports, the photocatalyst may be exposed to a gas such as air, or nitrogen, or oxygen. The flow of the gas across the photocatalyst sample may remove unwanted intermediates thereby maintaining optimal or near optimal photocatalytic performance.

In some embodiments, the photoreactor 100 may include at least three computer or microprocessor controlled input/output ports allowing for introduction of reactant CO₂ and H₂O (vapor) and collection of product. The photoreactor may be exposed to concentrated sunlight. In some embodiments, the concentrated sunlight may be at illumination intensities greater than 1 sun.

### EXAMPLE

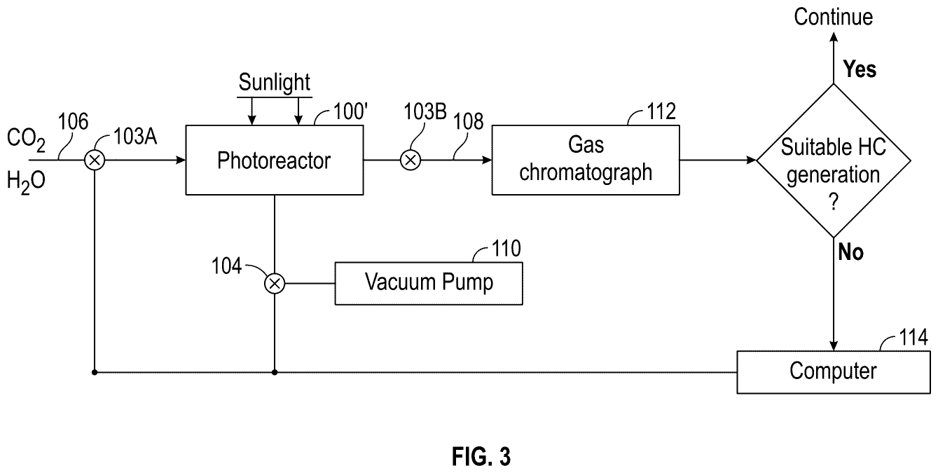

In an exemplary process diagramed in FIG. 3, the photoreactor 100′ is used for photoconversion of CO₂ and H₂O (vapor) feed 106 to hydrocarbon fuel product 108. The photoreactor 100′ includes three computer controlled valves or actuators: 103A controlling introduction of reactant, 103B controlling collection of product, and 104 connected to a vacuum pump 110. The product 108 is analyzed using a gas chromatograph 112. If the gas chromatograph 112 indicates suitable hydrocarbon generation, the process is continued. However, when the chromatograph 112 indicates reduced photoconversion, a computer 114 causes actuators 103A and 103B to close, actuator 104 to open, and the vacuum pump 110 is turned on to remove intermediate compounds that remain on the surface of the photocatalyst preventing adsorption of reactant molecules. After a pre-determined time, e.g., 60 s, actuator 104 is closed, the vacuum pump 110 is turned off, and actuators 103A and 103B are opened and photocatalysis is resumed

From the above description, it is clear that the inventive concepts disclosed and/or claimed herein are well adapted to satisfy the advantages mentioned herein, as well as those inherent in the present disclosure. While exemplary embodiments of the inventive concepts have been described for purposes of this disclosure, it will be understood that numerous changes may be made which will readily suggest themselves to those skilled in the art and which are accomplished within the spirit of the inventive concepts disclosed and/or claimed herein.

### REFERENCES CITED

S. Sorcar, Y. Hwang, J. Lee, H. Kim, K. M. Grimes, C. A. Grimes, J. W. Jung, C. H. Cho, T. Majima, M. R. Hoffmann, and Su-II In. CO₂, Water, and Sunlight to Hydrocarbon Fuels: A Sustained Sunlight to Fuel (Joule-to-Joule) Photoconversion Efficiency of 1%. Energy & Environmental Science 12 (2019) 2685-2696.
S. N. Habisreutinger, L. Schmidt-Mende, J. K. Stolarczyk, Photocatalytic reduction of CO₂ on TiO₂ and other semiconductors, Angewandte Reviews 52 (2013) 7372-7408.
S. C. Roy, O. K. Varghese, M. Paulose, C. A. Grimes, Toward solar fuels: photocatalytic conversion of carbon dioxide to hydrocarbons, ACS Nano 4 (2010) 1259-1278.

What is claimed is:

1. A method for photoconversion of CO₂ and H₂O vapor reactants to hydrocarbon fuel product, the method comprising:
introducing CO₂ and H₂O vapor reactants into a photoreactor and collecting hydrocarbon fuel product from the photoreactor using at least two computer or microprocessor-controlled input/output ports; and
varying the relative CO₂/H₂O concentration into the photoreactor as a function of time to maintain optimal or near optimal photocatalyst performance.

2. A method for photoconversion of CO₂ and H₂O vapor reactants to hydrocarbon fuel product, the method comprising:
introducing CO₂ and H₂O vapor reactants into a photoreactor and collecting hydrocarbon fuel product from the photoreactor using at least two computer or microprocessor-controlled input/output ports; and
modulating a photocatalyst temperature within the photoreactor as a function of time to maintain optimal or near optimal photocatalyst performance.

3. A method for photoconversion of CO₂ and H₂O vapor reactants to hydrocarbon fuel product, the method comprising:
introducing CO₂ and H₂O vapor reactants into a photoreactor and collecting hydrocarbon fuel product from the photoreactor using at least two computer or microprocessor-controlled input/output ports; and
modulating gas pressure within the photoreactor as a function of time to promote desorption of intermediate products from the photocatalyst to enable optimal or near optimal rates of product formation.

4. The method of claim 3, wherein the gas pressure within the photoreactor is modulated as a function of time while the gaseous flow rate through the photoreactor is kept constant.

5. The method of claim 4, further comprising using a computer or microprocessor-controlled mass flow controller, along with the computer or microprocessor-controlled input/output ports, to enable modulating the photoreactor gas pressure as a function of time, while keeping a constant gaseous flow rate through the photoreactor.

6. A method for operating a photoreactor having computer actuated input/output ports, the method comprising:
introducing reactant to the photoreactor through an input port and collecting product from the photoreactor through an output port; and
upon closure of the input and output ports, treating photocatalyst within the photoreactor to remove intermediates limiting performance of the photocatalyst;
resuming introduction of reactant to the photoreactor through the input port and resuming collection of product from the photoreactor through the output port.

7. The method of claim 6, wherein the step of treating the photocatalyst comprises passing a gas-phase atmosphere across ambient-temperature photocatalyst within the photoreactor for removal of intermediates limiting performance of the photocatalyst.

8. The method of claim 7, wherein the step of treating the photocatalyst further comprises heating the photocatalyst.

9. The method of claim 7, wherein the step of treating the photocatalyst further comprises exposure of the photocatalyst to ultraviolet-wavelength electromagnetic energy.

10. The method of claim 6, wherein the step of treating the photocatalyst comprises exposing the photocatalyst at ambient temperature to a vacuum for removal of intermediates from the photocatalyst surface.

11. The method of claim 6, wherein the step of treating the photocatalyst comprises exposing of the photocatalyst to a vacuum and holding the photocatalyst at a temperature elevated from ambient for breakdown and removal of intermediates limiting photocatalyst performance.

12. The method of claim 6, wherein the step of treating the photocatalyst comprises exposing the photocatalyst to a vacuum while illuminating the catalyst with electromagnetic energy for breakdown and removal of intermediates limiting photocatalyst performance.

13. The method of claim 6, wherein the step of treating the photocatalyst comprises exposing the photocatalyst to a vacuum while illuminating the catalyst with ultraviolet light for breakdown and removal of intermediates limiting photocatalyst performance.

14. The method of claim 6, wherein the photoreactor is exposed to concentrated sunlight with illumination intensities greater than 1 sun.

15. The method of claim 6, wherein the photoreactor comprises at least three computer or microprocessor-controlled input/output ports for introduction of reactant and collection of product, and wherein the step of treating the photocatalyst comprises exposing the photocatalyst to a gas-phase atmosphere is selected from the group consisting of air, argon, nitrogen, oxygen, and combinations thereof.

16. The method of claim 6, wherein the photoreactor comprises at least three computer or microprocessor-controlled input/output ports that allow for programmed iteration of system operation alternating between photocatalytic conversion of input reactant to output product and photocatalyst treatment to remove intermediates limiting performance of the photocatalyst.

17. The method of claim 16, wherein the photocatalyst treatment comprises introducing a liquid to the photoreactor to remove unwanted intermediates from the photocatalyst.

18. The method of claim 6, wherein the input and output ports are computer or microprocessor controlled, and upon closure of the input and output ports, the photocatalyst is treated by allowing desorption of the intermediates at ambient temperature.

19. The method of claim 18, wherein upon closure of the input and output ports, the photocatalyst is heated to desorb the intermediates.

20. The method of claim 18, wherein upon closure of the input and output ports, the photocatalyst is exposed to ultraviolet-wavelength electromagnetic energy to desorb the intermediates.
